(19) United States
(12) Patent Application Publication
(10) Pub. No.: US 2009/0131828 A1
(43) Pub. Date: May 21, 2009

(54) VACUUM SAMPLE EXPRESSION DEVICE

(76) Inventors: Daniel Wong, Sunnyvale, CA (US); Frank A. Chan, Sunnyvale, CA (US); Shilpa Schuettenhelm, Los Gatos, CA (US); John Kennedy, Santa Cruz, CA (US); Charles C. Raney, Camdenton, MO (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(21) Appl. No.: 12/355,910

(22) Filed: Jan. 19, 2009

Related U.S. Application Data
(63) Continuation of application No. 10/865,633, filed on Jun. 10, 2004, now abandoned.

(51) Int. Cl. A61B 5/151

(52) U.S. Cl. 600/578

(57) ABSTRACT

A body fluid sampling device is used to sample a bodily fluid from an incision in skin. The device includes a housing that defines a sample cavity with one open end that contacts skin. The device also includes a lancing mechanism that forms an incision in the skin. A vacuum mechanism is used to create a vacuum inside the sample cavity to express fluid from the incision site. To release the vacuum, a valve is connected with the sample cavity to exchange atmospheric air and further works to minimize fluid splatter. The fluid sample can then be tested or analyzed as desired.

### CROSS REFERENCE TO RELATED APPLICATIONS

This application is a continuation of application Ser. No. 10/865,633, filed Jun. 10, 2004, which is hereby incorporated by reference.

### BACKGROUND

The present invention generally relates to body fluid sampling devices and more specifically, but not exclusively, concerns an integrated body fluid sampling device that involves the use of a vacuum of and mechanical forces to assist in fluid expression from an incision site.

The acquisition testing of bodily fluids is useful for many purposes, and continues to grow in importance for use in medical diagnosis and treatment, and in other diverse applications. A common technique for collecting a bodily fluid sample is to form an incision in the skin to bring the fluid, such as blood or interstitial fluid, to the skin's surface. The fingertip is frequently used as the fluid source because it is highly vascularized and therefore produces a good quantity of blood. However, the fingertip also has a large concentration of nerve endings, and lancing the fingertip can therefore be painful. Alternate sampling sites, such as the palm of the hand, forearm, earlobe and the like, may be useful for sampling, and are less painful. However, they also produce lesser amounts of blood. These alternate sites therefore are generally appropriate for use only for test systems requiring relatively small amounts of fluid, or if steps are taken to facilitate the expression of the bodily fluid from the incision site. Thus, there is a need to be able to express fluid in a simple manner and assist expression from the incision site at those alternative body sites.

Thus, needs remain for further contributions in this area of technology.

### SUMMARY

One aspect of the present invention concerns a body fluid sampling device that includes a housing that defines a cavity with an open end adapted to contact skin. A lancing mechanism is disposed in the cavity to form an incision in the skin. The device further includes a vacuum mechanism to form a vacuum inside the cavity to express body fluid from the incision. A valve is disposed on the housing for releasing the vacuum in the cavity to minimize splatter of the body fluid when the vacuum is released.

Another aspect concerns a method of sampling body fluid. An open end of a body fluid sampling device is placed against skin. The sampling device includes a vacuum mechanism, an incision forming device and a vacuum release valve. An incision is formed in the skin with the incision forming device. A vacuum is created at the open end of the sampling device with the vacuum mechanism. Splattering of body fluid from the incision is minimized by releasing the vacuum with the vacuum release valve.

A further aspect concerns a body fluid sampling device that includes a housing that defines a cavity with an open end adapted to contact skin. A vacuum mechanism is connected with the cavity to form a vacuum inside the cavity. A piston is disposed in the cavity to apply mechanical force to skin.

Still yet another aspect concerns a method of sampling body fluid. A body fluid sampling device is placed against skin, and the body fluid sampling device includes a vacuum mechanism. A vacuum is created with the vacuum mechanism, and an incision is formed in skin. Mechanical force is applied against the skin with the sampling device to express body fluid from the incision.

Further forms, objects, features, aspects, benefits, advantages, and embodiments of the present invention will become apparent from a detailed description and drawings provided herewith.

### BRIEF DESCRIPTION OF THE DRAWINGS

FIG. 1 is a side cross sectional view of a body fluid expression device according to one embodiment of the present invention.

FIG. 2A is a side cross sectional view of a body fluid expression device according to another embodiment of the present invention.

FIG. 2B is a side cross sectional view of the FIG. 2A body fluid expression device in a lancing configuration.

FIG. 2C is a side cross sectional view of the FIG. 2A body fluid expression device in an expression configuration.

FIG. 3 is a side cross sectional view of a bodily fluid expression device according to a further embodiment of the present invention.

FIG. 4A is a side cross sectional view of a body fluid expression device according to another embodiment of the present invention.

FIG. 4B is a side cross sectional view of the FIG. 4A body fluid expression device in a primed configuration.

FIG. 4C is a side cross sectional view of the FIG. 4A body fluid expression device in a lancing configuration.

FIG. 5 is a side cross sectional view of a body fluid expression device according to a further embodiment of the present invention.

### DESCRIPTION OF SELECTED EMBODIMENTS

While the present invention may be embodied in many different forms, for the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

The present invention generally concerns a fluid expression device that mechanically creates a vacuum to express fluid. The device generally includes a sampling cap with one open end, a lancing mechanism to form an incision, a vacuum mechanism to express fluid, and a valve to release the vacuum. The open end of the sampling cap is placed onto the skin and a moderate force is applied to form a seal between the sampling cap and the skin. An incision is formed in the skin and then a vacuum mechanism creates a vacuum inside the sampling cavity to express fluid through the incision. It should be appreciated, however, that an incision could be formed in the skin before placing the device onto a section of skin. After the fluid is obtained, the air valve is opened to release the vacuum, thereby reducing the chance of fluid splattering inside the device. Additionally, a mechanical force can be used in conjunction with the vacuum to express fluid. The mechanical force is applied to the skin to concentrate fluid toward an expression site.

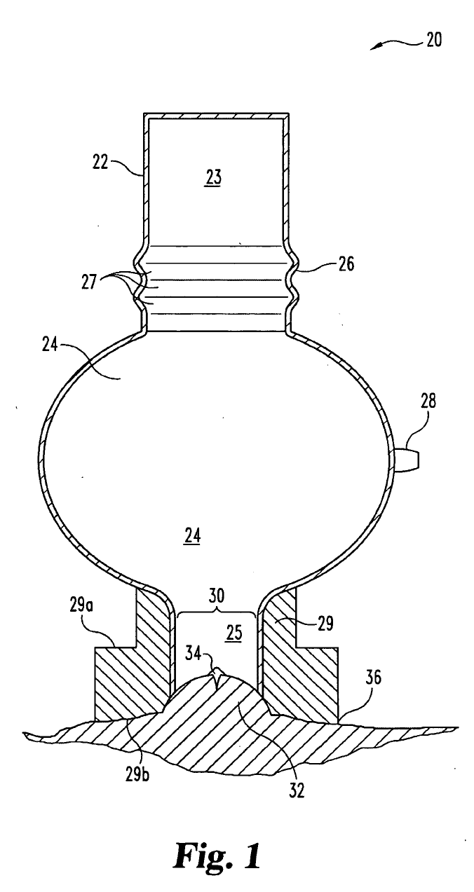

Referring to FIG. 1, a fluid expression or sampling device 20 according to one embodiment contains a housing 22 with different portions. One end of the housing 22 is a handle portion 23, which can serve as a handle for device 20 or can be used as a connecting portion to attach device 20 with other devices. For instance, the fluid expression device 20 can be integrated with a fluid sampling device that has a lancet and a test strip so that a complete fluid sampling procedure can be accomplished without the need to move the device 20. Another portion of the housing 22 defines a vacuum cavity 24 where a vacuum is formed. Vacuum cavity 24 is a generally spherical shape, but it should be appreciated that the cavity 24 can be shaped differently. Opposite handle portion 23 is expression portion 25. Another portion of the housing 22 includes an accordion section 26, for creating a vacuum, with a series of folds 27. In the illustrated embodiment, the accordion section 26 is located between the handle portion 23 and the vacuum cavity 24, and the vacuum cavity 24 is located in between the accordion section 26 and the expression portion 25. Moreover, it is envisioned that the accordion section 26 and the vacuum cavity 24 can be located at different locations on the device 20. It should be appreciated that the accordion section 26 can be any flexible collapsible mechanism with a series of folds made from plastic or other appropriate pliable material. The housing 22 also includes an air release valve 28 that communicates with the vacuum cavity 24. It should be understood that the valve 28 can be any type of one way control valve such as a check valve, for example. Moreover, it is contemplated that the valve 28 can be positioned at other locations on the device 20 in other embodiments. Surrounding the expression portion 25 is a base portion 29 with a flange 29a. The base portion 29 acts as a stabilizing member and a seal for the device 20. The flange 29a provides greater surface area for increased stabilization of device 20. It should be understood that the base portion 29 can be made of any appropriate flexible material that would create a seal with a section of skin. In one embodiment, the base portion 29 is made of rubber. The base portion 29 which surrounds the expression portion 25 contacts the skin 32 at surface 29b. At the expression portion 25, the housing 22 has an open end 30 that is configured to be placed over a section of the skin 32. The configuration of the expression portion 25 being open to the skin 32 reduces the distance fluid travels when it is collected. In this embodiment, the fluid expression device 20 has a generally cylindrical shape, but it should be understood that the device can be shaped differently as would occur to those skilled in the art.

Before operating the device 20, a lancing mechanism is used to create an incision 34 in the skin 32. The lancing mechanism can be incorporated into the device 20 or can be a separate device. It should be appreciated that any device used for forming an incision in the skin may be used, such as a needle or laser. The open end 30 and the base portion 29 are placed onto a section of the skin 32 and a moderate force is applied to form a seal 36 between the device 20 and the skin 32. It is contemplated that the force can be applied using a variety of methods including, but not limited to, mechanical mechanisms or a manually applied force. When force is continually applied to the fluid expression device 20 at the handle portion 23, the accordion section 26 collapses and expels air from the vacuum cavity 24 through the air valve 28. When the force is released, the accordion section 26 returns to normal state and creates a vacuum inside the vacuum cavity 24. The vacuum created inside the vacuum cavity 24 expresses fluid through the incision site 34. By repeatedly collapsing the accordion section 26, a greater vacuum can be formed inside the device 20, and additional fluid can be expressed from the incision 34. In the illustrated embodiment, after a sufficient amount of body fluid collects on the skin 32, the device 20 can be removed so that a test strip or some other type of testing device can be used to collect and analyze the fluid sample. As mentioned above, the expression device 20 can be incorporated into an integrated sampling device so that the lancing, expression and testing steps can be performed without the need of user intervention.

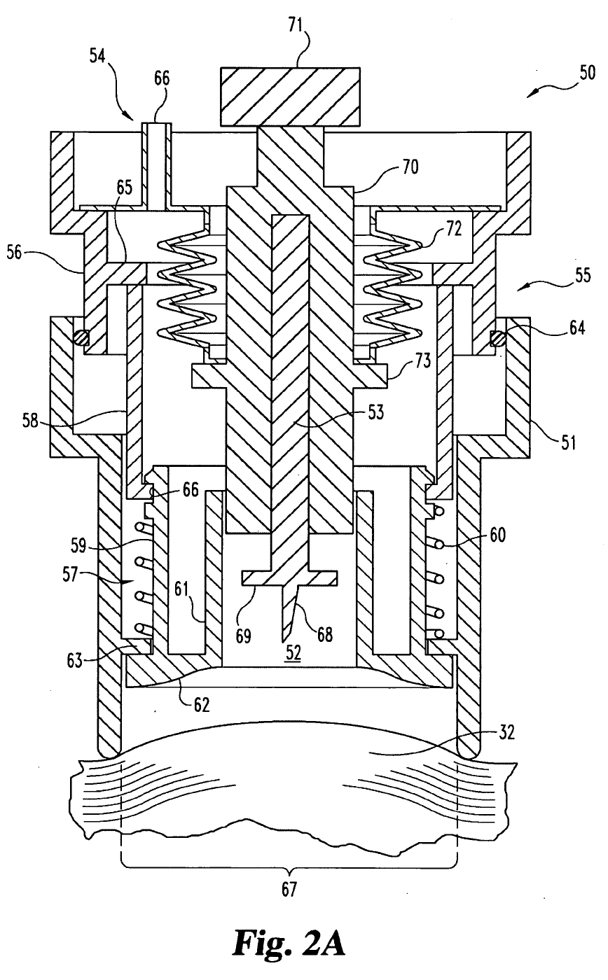

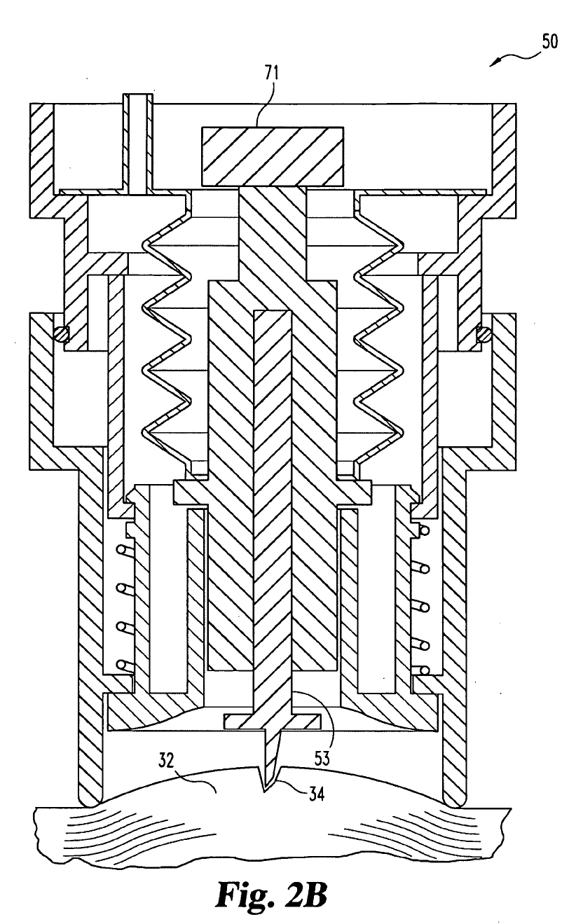

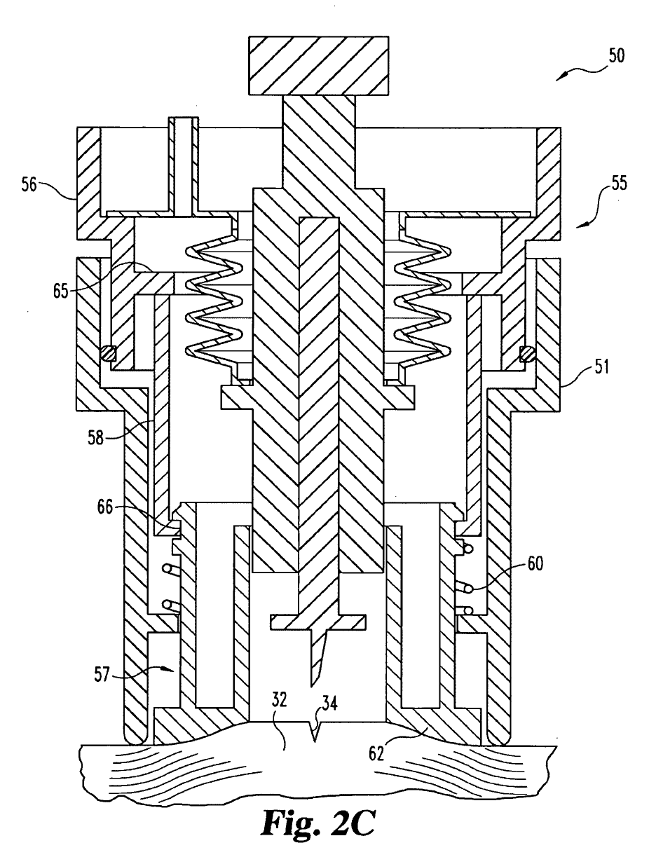

With reference to FIG. 2A, a sampling and expression device 50 according to another embodiment includes a housing 51 defining a sampling cavity 52 and a lancet 53 slidably disposed inside the housing 51. The lancet 53 is configured to form an incision 34 in the skin 32. The expression device 50 further includes a vacuum mechanism 54 to form a vacuum inside the expression device 50 so as to express fluid from the incision 34. As depicted, the device 50 further includes a piston mechanism 55 for applying mechanical force to the skin 32 to express fluid from the incision 34. As shown, the piston mechanism 55 includes an actuating portion 56 for initiating the piston mechanism 55, a skin contacting portion 57 for applying mechanical force to the skin 32, and a connecting portion 58 for operably connecting the actuating portion 56 and the skin contacting portion 57. The skin contacting portion 57 includes a spring carrier 59 which houses the spring 60, a lancet guide 61 for guiding the lancet 53 toward the skin 32 when activated, and an expression cap 62 designed to contact the skin 32.

At one end, the housing 51 is larger so as to receive the actuating portion 56 when the piston mechanism 55 is activated. The other end of the housing 51 is smaller so as not to receive the actuating portion 56 and configured to contact a section of the skin 32. As depicted, the housing 51 includes a flange 63, extending inwardly toward the sampling cavity 52. The flange 63 is designed to contact the expression cap 62 for stopping movement of the expression cap 62 inside the expression device 50. It is understood that the housing 51 can be formed from any type of appropriate material as would generally occur to those skilled in the art. In one embodiment, the housing 51 is made of plastic, but it should be appreciated that the housing 51 can be made of other materials. Similar to the design of housing 51, a section of the actuating portion 56 is smaller so as to be slidably received in the larger section of the housing 51, when the piston mechanism 55 is activated. A seal 64 maintains sealed contact between the housing 51 and the actuating portion 56. The actuating portion 56 also includes a flange 65 that is designed to contact an end of the connecting portion 58. At the other end, the connecting portion 58 contacts the skin contacting portion 57. Specifically, the end of connecting portion 58 that is proximal to the skin 32 is received into a notch 66 located on the spring carrier 59. The spring 60 is disposed around the spring carrier 59. As illustrated in FIG. 2A, the spring carrier 59 and the lancet guide 61 extend from the expression cap 62 in a direction away from the skin 32. Additionally, the lancet guide 61 and the spring carrier 59 are oriented parallel to one another. The lancet guide 61 is positioned closer to the lancet 53 and thus further inward toward the center of the expression device 50. It is understood that the piston mechanism 55, in other embodiments, can be configured differently and the components positioned at different locations within expression device 50.

The housing 51 further contains an air valve or vacuum opening 66, which operates as a vacuum source for the sampling cavity 52. In one embodiment, the air valve opening 66 is used as a connector for a tube from a vacuum pump. The vacuum pump is then used to create a vacuum inside the housing 51. In another embodiment, the air valve opening 66 includes a check valve to maintain the vacuum created inside the sampling cavity 52. Air is pumped from the housing 51 via the air valve opening 66 when the actuating portion 56 is compressed. The valve in this embodiment can be any type of one way control valve, such as a check valve. In the illustrated embodiment, the housing 51 includes an open end 67, which is placed onto a section of the skin 32. As shown, the fluid expression device 50 is oriented such that a portion of the skin 32 is received in the sampling cavity 52. In the illustrated embodiment, the fluid expression device 50 is generally cylindrically shaped. However, it should be understood that the device 50 can be differently shaped.

With reference to FIG. 2A, the fluid expression device 50 further includes the lancet 53 to create the incision 34 in a section of the skin 32. The lancet 53 includes a needle portion 68 that enters the skin 32 and creates the incision 34. A stop portion 69 is the maximum limit for penetration depth. In one embodiment, the depth control is integrated into the lancet 53. However, it should be appreciated that the depth control can be a separate system. The lancet 53 is contained in a lancet carrier 70 that surrounds the lancet 53. The lancet carrier 70 is coupled to a firing mechanism 71 that fires the lancet 53 into the skin 32. The firing mechanism 71 can include firing mechanisms of the type generally known in the art. For example, the firing mechanism 71 can include a spring, an electric motor and/or a pneumatic motor, to name a few. A flexible accordion component 72 seals against the piston mechanism 55, and on the other end, the accordion component 72 seals against the lancet carrier 70. As shown, the connection between the accordion 72 and the piston mechanism 55 is where the actuating portion 56 changes between a larger and smaller dimension. The accordion 72 operates as a seal to maintain the vacuum created inside the sampling cavity 52. In particular, the accordion component 72 allows the lancet 53 to move inside the sampling cavity 52 without disrupting the vacuum created. It should be appreciated that the flexible accordion component 72 can be any flexible collapsible mechanism with a series of folds made from plastic or other appropriate pliable material. Additionally, the lancet carrier 70 contains an extending flange member 73 which is designed to contact an end of the lancet guide 61 when the lancet 53 is activated.

To operate the fluid expression device 50, the open end 67 is placed onto a section of the skin 32. Force is then applied to the device 50 to form a seal between the housing 51 and the skin 32. The force can by manually applied by the user or automatically by the expression device 50. In the illustrated embodiment, the user presses the open end 67 of the expression device 50 against the skin 32 to form a seal. Once the seal is formed, the vacuum mechanism 54 is used to create a vacuum inside the sampling cavity 52. The vacuum mechanism 54 can be manually activated by the user or automatically started by the expression device 50. As mentioned before, the vacuum mechanism 54 in one embodiment includes a vacuum pump that is connected to valve opening 66 for creating a vacuum inside the sampling cavity 52. In the other previously mentioned embodiment, the vacuum is formed by manually pumping the device 50. Once formed, the vacuum created by the vacuum mechanism 54 bulges the skin 32 such that blood or other body fluids tend to be drawn to the incision site prior to lancing the skin 32. After the incision 34 is formed, the vacuum is then used to express fluid from the incision 34. In another embodiment, it is contemplated that the vacuum can be formed after the incision 34 is formed. In still yet another embodiment, the vacuum is formed before and after lancing the skin 32, but no vacuum exists when the skin is lanced. Nonetheless, it should be appreciated that the vacuum can be formed at other times, such as only when the skin 32 is lanced.

FIG. 2B illustrates the configuration of the fluid expression device 50 when the lancet 53 is creating the incision 34 in the skin 32. To fire the lancet 53, the firing mechanism 71 is manually triggered by the user or automatically triggered by the device 50. For example, once a sufficient vacuum is formed, the expression device 50 can automatically fire the lancet 53. Once fired, the firing mechanism 71 pushes the lancet 53 down into the skin 32 to form the incision 34, and the lancet guide 61 guides the lancet 53 toward the skin 32. Although the fluid expression device 50 is shown as incorporating the lancet 53, it should be appreciated that in other embodiments the lancet 53 can be separate from the expression device 50. Thus, the incision 34 can be formed either before or after the expression device 50 is placed onto the skin 32. After forming the incision 34, the firing mechanism 71 retracts the lancet 53 from the skin 32. It is contemplated that in other embodiments the lancet 53 can temporarily remain in the incision 34 after it is formed to brace open the incision 34 so as to promote blood flow from the incision.

With reference to FIG. 2C, after the incision 34 is formed, the expression cap 62 pressed against the skin 32 to express fluid from the incision 34. The pressure applied by the expression cap 62 against the skin 32, in conjunction with the vacuum created by the vacuum mechanism 54, enhances the fluid flow from the incision 34. The contact between the expression cap 62 with the skin 32 aids in fluid expression by concentrating fluid flow towards the incision 34. In one embodiment, the user manually presses the piston mechanism 55 such that the expression cap 62 is pressed against the skin. However, it is contemplated that the piston mechanism can be automatically actuated with a motor. To operate the piston mechanism 55, initially a force is applied to the actuating portion 56 in a direction toward the skin 32, for example by the user pressing against the actuating portion 56. Simultaneously, the flange 65, connected to the actuating portion 56, transmits the force to the connecting portion 58. As shown, the connecting portion 58 and the skin contacting portion 57 are operatively connected together at the notch 66. As a result of the force being applied to the actuating portion 56, the skin contacting portion 57 is also pushed down toward the skin 32, thereby compressing the spring 60. Eventually, the expression cap 62 of the skin contacting portion 57 presses against the skin 32. When pressed against the skin 32, the expression cap causes body fluid to be discharged from the incision 34. As mentioned above, the combined mechanical pressure and the vacuum, enhance expression of fluid from the incision 34. If the piston mechanism 55 is pressed further, the smaller section of the actuating portion 56 contacts the larger section of the housing 51 so that the movement of the piston mechanism 55 is stopped. When the applied force is removed, the spring mechanism 60 retracts the expression cap 62 from the skin 32 back to a configuration in which the expression cap 62 contacts the flange 63 extending from the housing 51, as is shown in FIG. 2A. It should be appreciated that the piston mechanism 55 can be repeatedly pressed to create a pumping action so as to express additional fluid can be expressed.

In the illustrated embodiment, fluid is expressed from the incision 34 as a result of the combined vacuum force and the mechanical force applied from the piston mechanism 55. It should be appreciated that the mechanical and vacuum forces can be applied in a different sequence or duration, if desired. After a sufficient amount of body fluid collects on the skin 32 in the illustrated embodiment, the device 50 can be removed so that a test strip or some other type of testing device can be used to collect and analyze the fluid sample. However, the expression device 50 can also be incorporated into an integrated sampling device so that the lancing, expression and testing steps can be performed without the need of user intervention. The body fluid can be tested for various medical properties, such as blood glucose levels.

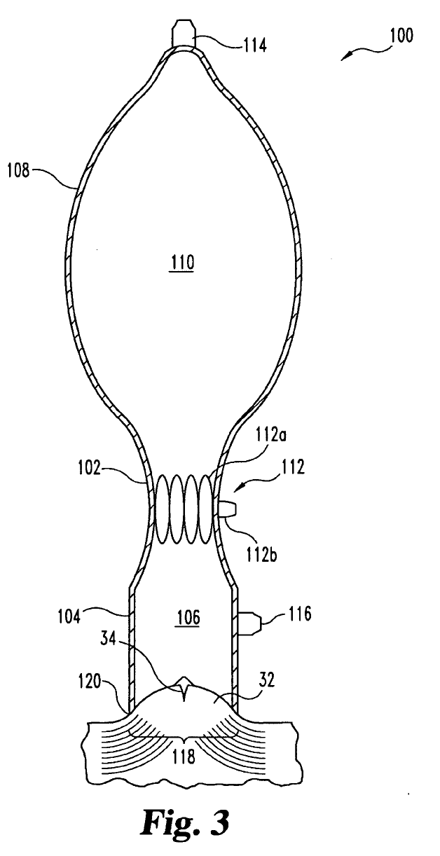

A body fluid expression device 100 according to another embodiment of the present invention is illustrated in FIG. 3. The fluid expression device 100 of this embodiment includes a housing 102. One end of the housing 102 includes an expression portion 104 defining a sampling cavity 106 where fluid is collected. Another portion of the housing 102 includes a flexible portion or bladder 108 defining a vacuum cavity 110 where a vacuum is created. The device 100 in one embodiment is made of plastic. However, it is understood that the housing 102 can be formed from any type of appropriate material as would generally occur to those skilled in the art. As should be appreciated, the flexible portion 108 can include any type of collapsible bladder mechanism made from plastic or other appropriate pliable material. The bladder 108 has a generally oval cross section, however it should be appreciated that the bladder 108 could be shaped differently.

Referring to FIG. 3, the bladder 108 and the expression portion 104 are separated by a vacuum valve system 112. The vacuum valve system 112 includes a compressible section 112a and a valve 112b. It is however contemplated that the bladder 108, expression portion 104, and vacuum valve system 112 can be located at different locations in other embodiments. The bladder 108 contains an air release valve 114. A vacuum release valve 116 positioned on the housing 102 and operates to release the vacuum formed inside the sampling cavity 106. It should be appreciated that for valves 114, 116, and 112b, any types of appropriate one way release valves can be used as known by those skilled in the art, such as check valves for example. Additionally, it is contemplated that the valves 114, 116, and 112b can be located at other locations on the device 100 in other embodiments. The housing 102 includes an open end 118 configured to be placed over a section of skin 32. The sampling cavity 106 is positioned in between the vacuum valve system 112 and the open end 118. The configuration of the sampling cavity 106 being open to the skin 32 reduces the distance the body fluid sample must travel when it is collected and allows the body fluid sample to be expressed under vacuum conditions. The vacuum is released through the valve 116 to reduce fluid splattering. In this embodiment, the fluid expression device 100 has a generally cylindrical shape, but it is contemplated that the device can be shaped differently.

In the illustrated embodiment, the device 100 does not include a lancing mechanism. For the illustrated embodiment, a separate lancing device is used to form the incision 34, and after the incision 34 is formed, the expression device 100 is placed over the incision 34 to express fluid. Nevertheless, it is envisioned that the lancing mechanism can be incorporated into the expression device 100 such that user can lance the skin and express fluid in a single operation. Referring again to FIG. 3, after the incision 34 is formed, the open end 118 of fluid expression device 100 is placed over the incision 34, and the user manually presses the expression device 100 against the skin 32 to create a seal 120 between the housing 102 and the skin 32. As should be appreciated, the expression device 100 can be configured so that the device is automatically pressed against the skin 32, such as via a motor. Once the expression device 100 seals against the skin 32, the bladder 108 is compressed such that air is expelled through the air release valve 114. It is further contemplated that the bladder 108 can be compressed before the device 100 is placed onto the skin 32, and the bladder 108 can be manually or automatically compressed. The bladder 108 is then released, and due to the resilient nature of the bladder 108, the bladder 108 attempts to return to its original shape, which in turn creates a vacuum inside the sampling cavity 106. Continuing depression of the vacuum valve system 112 expresses fluid through incision point 34. As shown, section 112a has as a set of flexible ribs that prevent section 112a from collapsing. The vacuum formed in the sampling cavity 106, in combination with a continuing the moderate force applied on the device 100 in a direction towards the skin 32, expresses fluid from the incision 34. After the desired amount of fluid is expressed, the user presses valve 116 to gradually release the vacuum in the sampling cavity 106. The valve 116 gradually releases the vacuum so as to minimize splattering of fluid within the sampling cavity 106. As should be appreciated, the reduction the splattering of body fluid reduces waste and generally promotes more hygienic conditions. After releasing the vacuum, the device 100 can then be removed from the skin 32 so that a test strip or some other type of testing device can be used to collect and analyze the fluid sample. In another embodiment, the expression device 100 incorporates into an integrated sampling device so that the lancing, expression and testing stages can be performed without the need of user intervention.

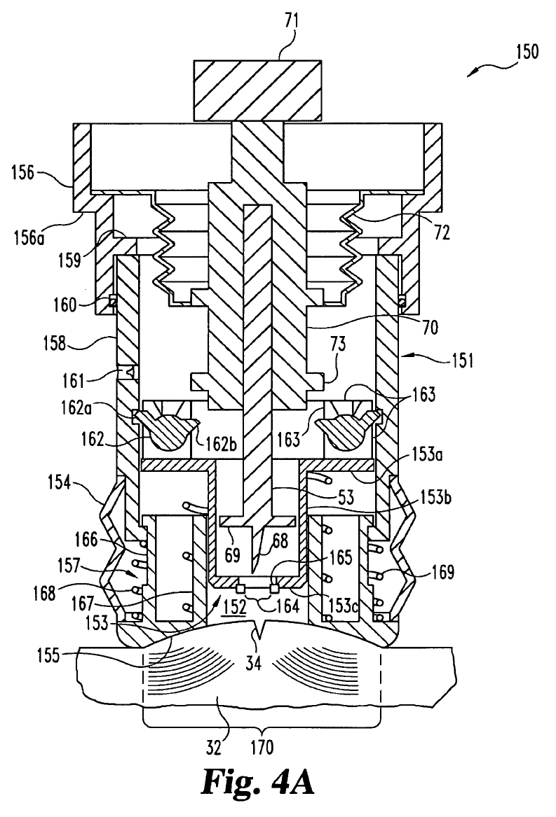

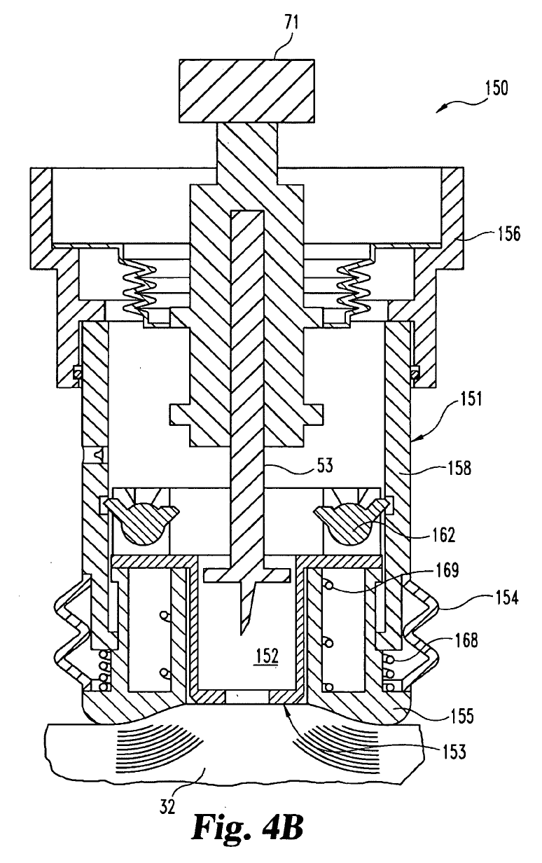

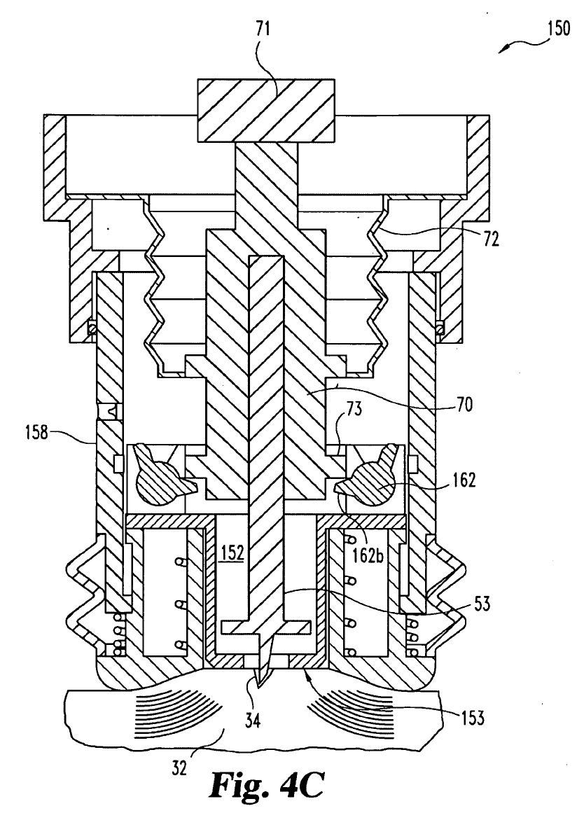

Referring now to FIG. 4A, a fluid expression device 150 according to another embodiment includes a housing 151 that defines a sampling cavity 152 and a lancet 53 slidably disposed inside the housing 151. The lancet 53 is configured to form an incision 34 in the skin 32. A lancing cap 153 is configured to control penetration depth of lancet 53 into the skin 32 by flattening the skin 32 around the lancet during lancing. The expression device 150 further includes a bellow section 154 that is used to form a vacuum inside the sampling cavity 152 to express fluid from the incision 34 formed by the lancet 53. In the illustrated embodiment, the housing 151 includes an expression cap 155 that is pressed against the skin 32 to assist in expressing fluid from the incision 34.

As mentioned above, the fluid expression device 50 has the lancet 53 to create the incision 34 in the skin 32. The lancet 53 includes a needle portion 68 that enters the skin 32 and creates the incision 34. The lancet 53 further includes a stop portion 69 that is designed to contact the contact portion 153c of lancing cap 153 to control maximum penetration depth. The lancet 53 is contained in the lancet carrier 70, which surrounds the lancet 53. As depicted, the lancet firing mechanism 71 is used to fire the lancet carrier 70, at a pre-determined penetration depth setting, so as to form the incision 34 at a desired penetration depth. Flexible accordion component 72 seals between the actuation portion 156 and the lancet carrier 70 so as to maintain a vacuum within the sampling cavity 152 of the device 150. This allows the lancet 53 to move inside the sampling cavity 152 without disrupting the vacuum created. It should be appreciated that the accordion component 72 can be any flexible collapsible mechanism with a series of folds made from plastic or other appropriate pliable material. A flange 73 extends from the lancet carrier 70.

As depicted, the housing 151 includes an actuating portion 156, which is used to create a vacuum and press the expression cap 155 against the skin 32 to express fluid. In particular, the housing 151 has a skin contacting portion 157 that is configured to press against the skin 32 to express body fluid. The device 150 further includes a connecting portion 158 that operatively couples the actuating portion 156 to both the bellow section 154 and to the skin contacting portion 157. With reference to FIG. 4A, the bellows section 154 forms a seal between the skin contacting portion 157 and the connecting portion 158. It should be understood that the bellow section 154 can, for example, include any type of collapsible structure with a series of folds made from plastic or other appropriate pliable material. As depicted, the actuating portion 156 is larger at an end distal to the skin 32, and a step portion 156a defines the transition from the smaller section to the larger section of the actuating portion 156. The actuating portion 156 further includes a flange 159, which extends radially inward toward the center of expression device 150. The flange 159 acts as a stop for the actuating portion 156 when contacting the connecting portion 158. Additionally, a seal 160 is located between the actuating portion 156 and the connecting portion 158 within the expression device 150. The seal 160 assists in maintaining the vacuum created inside the sampling cavity 152. The connecting portion 158 incorporates an air valve 161, which allows air to leave the sampling cavity 152 so as to create a vacuum inside the sampling cavity 152. It should be appreciated that the valve 161 can include any type of one way control valve, such as a check valve.

A shown in FIG. 4A, the lancing cap 153 has one or more cams 162 that are used to detachably secure the lancing cap 153 to the connecting portion 158. A first extension 162a of the cam 162 engages a notch in the connecting portion 158 to secure the lancing cap 153 to the connecting portion 158 so that the lancing cap 153 and the connecting portion are able to move in unison. A second extension 162b of the cam 162 extends radially inwards toward the center of fluid expression device 150. The second extension 162b extends from the cam 162 so as to be able to engage the flange 73 on the lancet 70. As depicted, the cams 162 are pivotally coupled to the lancing cap 153 via a cam support member 163 on the lancing cap 153. In one embodiment, the cams 162 include springs that bias the cams 162 into an orientation in which the first extensions 162a engage the notch in the connecting portion 158. When the lancet 70 is fired, the flange 73 on the lancet 70 engages the second extension 162b on cam 162 such that the cam 162 rotates, thereby disengaging the first extension 162a from the notch in the connecting portion 158. Once disengaged, the lancing cap 153 is able to move independently of the connecting portion 158 such that the lancing cap 153 is able to retract from the skin 32 upon retraction of the lancet 70. Lancing cap 153 includes a flange portion 153a, a body portion 153b, and a skin contact portion 153c. As shown, the cam support member 163 extends from the flange portion 153a, and the body portion 153b connects the flange portion 153a with the skin contact portion 153c. The contact portion 153c is positioned to contact the skin 32, and the skin contact portion 153c defines an aperture 164 through which the lancet 70 extends during lancing. Around the aperture 164, the skin contact portion 153c has a seal 165 for sealing against the skin 32 to maintain skin surface uniformity. It should be appreciated that the seal 165 can be any type of seal, such as an O-ring seal for example.

As mentioned before, the connecting portion 158 and the skin contacting portion 157 are connected by the bellow section 154. In addition, the expression cap 155 has a groove to which a flange from the connecting portion 158 is slidably disposed. As shown, the expression cap 155 has a lancing cap guide 167 that guides that guides the lancing cap 153 during lancing. A biasing spring 168 for biasing the skin contacting portion 157 from the connecting portion 158 is positioned between the skin contacting portion 157 of the expression cap 155 and the connecting portion 158. Around the cap guide 167, a retraction spring 169 is disposed that biases the skin contact portion 153c of the lancing cap 153 from the skin 32. Referring to FIG. 4A, the housing 151 includes an open end 170, which is placed onto a section of the skin 32. The fluid expression device 150 is oriented such that the skin 32 is open to the sampling cavity 152. In the illustrated embodiment, the fluid expression device 150 is generally cylindrically shaped. However, it should be understood that the device 150 could be configured differently.

To operate the fluid expression device 150, the open end 170 is placed onto the skin 32. In the illustrated embodiment, the user presses the device 150 against the skin 32 to form a seal between the housing 151 and the skin 32. It should be understood that the device 150 in other embodiments can be automatically pressed against the skin 32. The pressing force is applied to the actuating portion 156, which transmits a force to the connecting portion 158 as a result of the contact between the flange 159 with the connecting portion 158. This force then compresses the bellow section 154 and expels air through the valve 161. As the device 150 is pressed against the skin 32, both springs 168, 169 are compressed, and the bellow section 154 continues to collapse until the lancing cap 153 contacts the skin 32, as is shown in FIG. 4B. Once the device 150 is no longer pressed further against the skin 32, the now compressed biasing spring 168 pushes the connecting portion 158 away from the skin 32, thereby creating a vacuum inside the sampling cavity 152. The vacuum created inside the sampling cavity 152 aids in priming the incision site by drawing body fluid in the skin 32 towards the incision site. As shown in FIG. 4B, the lancing cap 153 flattens the skin 32 around the incision site in preparation of lancing of the skin 32. The seal 165 on the lancing cap 153 helps to keep the skin tight during lancing so as to reduce the variability in the penetration depth of the lancet 53.

Referring to FIG. 4C, when the lancet 53 is fired by the firing mechanism 71, either by being manually or automatically triggered, the lancet 53 penetrates the skin 32 to form an incision 34. As mentioned before, the lancing cap 153 flattens the skin 32 around the lancet 53 to minimize bulging of the skin 32, which reduces the variability in the penetration depth of the lancet 53. During lancing, the accordion section 72 assists in maintaining a vacuum inside the device 150. As the lancet 53 is fired, the flange 73 on the lancet carrier 70 rotates the cam 162 by engaging the second extension 162b on the cam 162, thereby disengaging the lancing cap 153 from the notch in the connecting portion 158. After the incision 34 is formed, the firing mechanism 71 retracts the lancet 53 from the skin 32. Since the lancing cap 153 is disengaged from the connecting portion 158, the retraction spring 169 is able to retract the lancing cap 153 from the skin 32. By retracting the lancing cap 153, a larger opening is formed in which body fluid is expressed from the incision. With the larger opening size, a greater amount of fluid can be expressed from the incision, and the risk of smearing the fluid sample is minimized. To increase the vacuum in the device 150, the actuation portion 156 can be pulled away from the skin 32 such that the cams 162 re-engage the connecting portion 158, as is shown in FIG. 4A. The now larger volume of cavity 152 creates an even lower pressure, which can further enhance expression of fluid from the incision 34.

Fluid is expressed from the incision 34 as a result of the combined vacuum force and mechanical force. To express fluid, the device 150 can be pressed against the skin 150 to force fluid out of the incision 34. Referring to FIG. 4A, the pressing action of the expression cap 155 causes the fluid to concentrate in the incision 34. It should be appreciated that the mechanical and vacuum forces can be applied in a different sequence or duration if desired. Additionally, it should be appreciated that the bellow section 154 can be activated numerous times over the incision site 34. By repeatedly compressing the bellow section 154 and creating a pumping action in concentrating fluid toward the incision 34, additional fluid can be expressed. In the illustrated embodiment, after a sufficient amount of body fluid collects on the skin 32, the valve 161 is gradually opened to minimize splattering of body fluid in the device 150. Once the pressure has equalized, the device 150 can be removed from the skin 32, and a test strip, capillary tube or some other collection means can be used to collect and analyze the fluid sample. In another embodiment, the expression device 150 is incorporated into an integrated sampling device that has a test device so that the lancing, expression and testing stages can be performed without the need of user intervention.

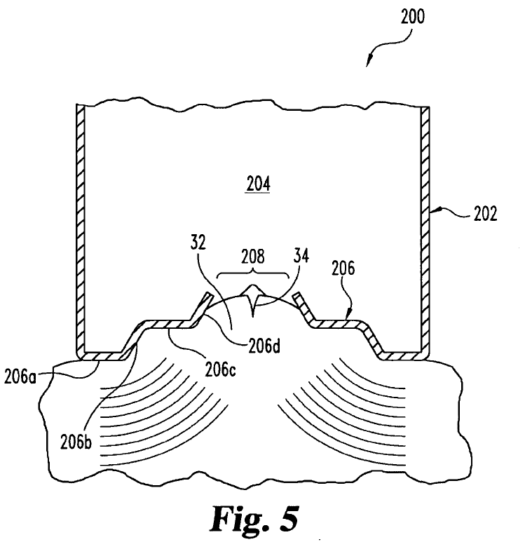

Referring to FIG. 5, a fluid sample expression device 200 according to another embodiment is illustrated. The device 200 includes a housing 202, which defines a sampling cavity 204 where fluid is collected. As will be appreciated from the discussion below, the expression device 200 of FIG. 5 can be incorporated into the devices described above. The housing 202 is formed from any type of appropriate material as would generally occur to those skilled in the art, such as plastic. In the illustrated embodiment, the fluid expression device 200 has a generally cylindrical shape, but it should be understood that the device 200 can be shaped differently in other embodiments. One end of the housing 202 has an expression surface 206 in the form of a ring, which is configured to be placed onto a section of skin 32. The expression surface 206 has an aperture 208 through which an incision is formed. As shown, the expression surface 206 is shaped in a stair step fashion to enhance fluid expression by concentrating fluid at the incision 34. In FIG. 5, the expression surface 206 has an outer radial surface 206a, and an outer radial wall 206b connected to the outer radial surface 206a. In the illustrated embodiment, the outer radial surface 206a extends in a general radially inward manner, and the outer radial wall 206b has a frustoconical shape that extends into the sampling cavity 204. An inner radial surface 206c extends in a general radially inward manner from the outer radial wall 206b. The inner radial surface 206c is connected to an inner radial wall 206d that extends further inside the sampling cavity 204 and defines the sampling cavity 204. In the illustrated embodiment, the inner radial wall 206d has a frustoconical shape. As can be seen, the surfaces 206a, 206c and the walls 206b, 206d give the expression surface the overall stair-Stepped shape, which enahances fluid expression. The shape of the expression surface 206 enhances expression of fluid when the device 200 is pressed against the skin and/or when a vacuum is used to express body fluid.

All publications and patent applications cited in this specification are herein incorporated by reference as if each individual publication or patent application were specifically and individually indicated to be incorporated by reference. Further, any theory, mechanism of operation, proof, or finding stated herein is meant to further enhance understanding of the present invention, and is not intended to limit the present invention in any way to such theory, mechanism of operation, proof, or finding. While the invention has been illustrated and described in detail in the drawings and foregoing description, the same is to be considered as illustrative and not restrictive in character, it being understood that only selected embodiments have been shown and described and that all equivalents, changes, and modifications that come within the spirit of the inventions as defined herein or by the following claims are desired to be protected.

What is claimed is:

1. A method, comprising:
placing a body fluid sampling device against skin, wherein the body fluid sampling device includes a sampling cavity, a vacuum mechanism, a lancing mechanism with a lancet, and a piston slidably disposed inside the sampling cavity;
creating a vacuum inside the sampling cavity with the vacuum mechanism;
forming an incision in the skin by extending the lancet into the skin and retracting the lancet from the incision with the lancing mechanism;
pressing the piston of the sampling device against the skin after said forming the incision to express body fluid from the incision by extending the piston inside the sampling cavity against the skin while the vacuum mechanism applies the vacuum in the sampling cavity; and
collecting the body fluid from the surface of the skin with the lancet retracted from the incision.

2. The method of claim 1, wherein said creating the vacuum occurs before said forming the incision.

3. The method of claim 1, wherein said creating the vacuum occurs after said forming the incision.

4. The method of claim 1, wherein said forming the incision occurs before said placing the body fluid sampling device against the skin.

5. The method of claim 1, further comprising testing the body fluid with the sampling device.

6. The method of claim 1, further comprising controlling the depth of the incision in the skin.

7. The method of claim 6, wherein said controlling the depth of the incision in the skin includes flattening the skin with a lancing cap.
